(19) United States
(12) Patent Application Publication
(10) Pub. No.: US 2013/0137775 A1
(43) Pub. Date: May 30, 2013

(54) COMPOSITIONS AND METHODS FOR THE TREATMENT OF SYSTEMIC AA AMYLOID DISEASES

(71) Applicants: Alan D. Snow, Lynnwood, WA (US); Thomas Lake, Snohomish, WA (US); Luke Esposito, Seattle, WA (US); Kelsey Hanson, Seattle, WA (US); Marisa-Claire Yadon, Bellevue, WA (US); Joel Cummings, Seattle, WA (US)

(72) Inventors: Alan D. Snow, Lynnwood, WA (US); Thomas Lake, Snohomish, WA (US); Luke Esposito, Seattle, WA (US); Kelsey Hanson, Seattle, WA (US); Marisa-Claire Yadon, Bellevue, WA (US); Joel Cummings, Seattle, WA (US)

(21) Appl. No.: 13/747,828

(22) Filed: Jan. 23, 2013

Related U.S. Application Data
(63) Continuation-in-part of application No. 13/413,417, filed on Mar. 6, 2012.
Application No. 13/413,417 is a division of application No. 12/837,721, filed on Jul. 16, 2010, now Pat. No. 8,163,957.
Application No. 12/837,721 is a continuation of application No. 12/269,017, filed on Nov. 11, 2008, now abandoned.
Application No. 12/269,017 is a continuation of application No. 10/452,851, filed on May 30, 2003, now Pat. No. 7,514,583.
(60) Provisional application No. 61/592,117, filed on Jan. 30, 2012.
(60) Provisional application No. 60/385,144, filed on May 31, 2002.
(60) Provisional application No. 60/409,100, filed on Sep. 9, 2002.
(60) Provisional application No. 60/412,272, filed on Sep. 20, 2002.
(60) Provisional application No. 60/435,880, filed on Dec. 20, 2002.
(60) Provisional application No. 60/463,104, filed on Apr. 14, 2003.

(51) Int. Cl. C07C 235/56

(52) U.S. Cl. CPC C07C 235/56
USPC 514/622

(57) ABSTRACT

Bis- and tris-dihydroxyaryl compounds their synthesis, pharmaceutical compositions containing them, and their use in the treatment of amyloid diseases, especially AA amyloidosis, and the manufacture of medicaments for such treatment.

### RELATED APPLICATIONS

This application claims priority under 35 USC 119(e) to U.S. Provisional Application No. 61/592,117 filed Jan. 30, 2012.

This application is a continuation-in-part of U.S. application Ser. No. 13/413,417 filed Mar 6, 2012 which is a continuation-in-part of 12/837,721 filed Jul. 16, 2010, now U.S. Pat. No. 8,163,957, issued on Apr. 24, 2012, which claimed the benefit of priority under 35 U.S.C. §120 to, and was a continuation of U.S. application Ser. No. 12/269,017, filed Nov. 11, 2008, now abandoned, which is a continuation of U.S. application Ser. No. 10/452,851, filed May 30, 2003, now a U.S. Pat. No. 7,514,583, issued on Apr. 7, 2009, which claims priority under 35 USC 119(e) to:
(1) U.S. Provisional Application No. 60/385,144, filed May 31, 2002,
(2) U.S. Provisional Application No. 60/409,100, filed Sep. 9, 2002,
(3) U.S. Provisional Application No. 60/412,272, filed Sep. 20, 2002,
(4) U.S. Provisional Application No. 60/435,880, filed Dec. 20, 2002, and
(5) U.S. Provisional Application No. 60/463,104, filed Apr. 14, 2003.

The entire contents of all of these applications are incorporated by reference into this application.

### TECHNICAL FIELD

This invention relates to dihydroxyaryl compounds, their synthesis, pharmaceutical compositions containing them, and their use in the treatment of amyloid diseases, especially AA amyloid disease, and in the manufacture of medicaments for such treatment.

### BACKGROUND OF THE INVENTION

Alzheimer's disease is characterized by the accumulation of a 39-43 amino acid peptide termed the β-amyloid protein or Aβ, in a fibrillar form, existing as extracellular amyloid plaques and as amyloid within the walls of cerebral blood vessels. Fibrillar Aβ amyloid deposition in Alzheimer's disease is believed to be detrimental to the patient and eventually leads to toxicity and neuronal cell death, characteristic hallmarks of Alzheimer's disease. Accumulating evidence implicates amyloid, and more specifically, the formation, deposition, accumulation and/or persistence of Aβ fibrils, as a major causative factor of Alzheimer's disease pathogenesis. In addition, besides Alzheimer's disease, a number of other amyloid diseases involve formation, deposition, accumulation and persistence of Aβ fibrils, including Down's syndrome, disorders involving congophilic angiopathy, such as but not limited to, hereditary cerebral hemorrhage of the Dutch type, inclusion body myositosis, dementia pugilistica, cerebral β-amyloid angiopathy, dementia associated with progressive supranuclear palsy, dementia associated with cortical basal degeneration and mild cognitive impairment.

A variety of other human diseases also demonstrate amyloid deposition and usually involve systemic organs (i.e. organs or tissues lying outside the central nervous system), with the amyloid accumulation leading to organ dysfunction or failure. These amyloid diseases (discussed below) displaying marked amyloid accumulation in a number of different organs and tissues, and are known as systemic amyloidoses. In systemic AA amyloid disease, there is currently no cure or effective treatment, and the patient usually dies within 3 to 10 years from disease onset.

Systemic amyloidoses which include the amyloid associated with chronic inflammation, various forms of malignancy and familial Mediterranean fever (i.e. AA amyloid or inflammation-associated amyloidosis) (Benson and Cohen, Arth. Rheum. 22:36-42, 1979; Kamei et al, Acta Path. Jpn. 32:123-133, 1982; McAdam et al., Lancet 2:572-573, 1975; Metaxas, Kidney Int. 20:676-685, 1981), are known to involve amyloid deposition in a variety of different organs and tissues generally lying outside the central nervous system. Amyloid deposition in these diseases may occur, for example, in liver, heart, spleen, gastrointestinal tract, kidney, skin, and/or lungs (Johnson et al, N. Engl. J. Med. 321:513-518, 1989). For most of these amyloidoses, there is no apparent cure or effective treatment and the consequences of amyloid deposition can be detrimental to the patient. For example, amyloid deposition in the kidney may lead to renal failure, whereas amyloid deposition in the heart may lead to heart failure. For these patients, amyloid accumulation in systemic organs leads to eventual death generally within 3-5 years.

### SUMMARY OF THE INVENTION

In a first aspect, this invention is a dihydroxyaryl compound in a SMEDD formulation and pharmaceutically acceptable esters, and pharmaceutically acceptable salts thereof. The compounds are useful in the treatment of systemic AA amyloid diseases.

The compound 3,4-dihydroxybenzoic acid 3,4-dihydroxyanilide (compound 51) and pharmaceutically acceptable salts of the compound.

In a second aspect, this invention is pharmaceutical compositions comprising 3,4-dihydroxybenzoic acid 3,4-dihydroxyanilide and pharmaceutically acceptable excipients such as oils and surfactants.

In a third aspect, this invention is a method of treating a systemic AA amyloid disease in a mammal, especially a human, by administration of a therapeutically effective amount of a compound of the first aspect of this invention, for example as a pharmaceutical composition.

In a fourth aspect, this invention is the use of a compound of the first aspect of this invention in the manufacture of a medicament for the treatment of a systemic AA amyloid disease.

### BRIEF DESCRIPTION OF THE DRAWINGS

FIG. 1 is a graph of illustrating that a compound of the invention causes inhibition of of AA amyloid formation in mouse liver as assessed by Congo Red Fluorescence.

FIG. 2 is a graph of illustrating that a compound of the invention causes inhibition of of AA amyloid formation in mouse kidney as assessed by Congo Red Fluorescence.

FIG. 3 is a graph of illustrating that a compound of the invention causes inhibition of of AA amyloid formation in mouse spleen as assessed by Congo Red Fluorescence.

FIG. 4 is a graph of illustrating that a compound of the invention in a SMEDDS formulation causes inhibition of of AA amyloid formation in mouse liver as assessed by anti-AA Immunostaining.

FIG. 5 is a graph of illustrating that a compound of the invention in a SMEDDS formulation causes inhibition of of AA amyloid formation in mouse kidney as assessed by anti-AA Immunostaining.

### DETAILED DESCRIPTION OF THE INVENTION

### Definitions

In this application, the following terms shall have the following meanings, without regard to whether the terms are used variantly elsewhere in the literature or otherwise in the known art.

“Mammal” includes both humans and non-human mammals, such as companion animals (cats, dogs, and the like), laboratory animals (such as mice, rats, guinea pigs, and the like) and farm animals (cattle, horses, sheep, goats, swine, and the like).

“Pharmaceutically acceptable excipient” means an excipient that is conventionally useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

A “therapeutically effective amount” in general means the amount that, when administered to a subject or animal for treating a disease, is sufficient to affect the desired degree of treatment for the disease. A “therapeutically effective amount” or a “therapeutically effective dosage” preferably inhibits, reduces, disrupts, disassembles amyloid or synuclein fibril formation, deposition, accumulation and/or persistence, or treats a disease associated with these conditions, such as an amyloid disease or a synucleinopathy, by at least 20%, more preferably by at least 40%, even more preferably by at least 60%, and still more preferably by at least 80%, relative to an untreated subject. Effective amounts of a compound of this invention or composition thereof for treatment of a mammalian subject are about 0.1 to about 1000 mg/Kg of body weight of the subject/day, such as from about 1 to about 100 mg/Kg/day, especially from about 10 to about 100 mg/Kg/day. A broad range of disclosed composition dosages are believed to be both safe and effective.

“Treating” or “treatment” of a disease includes preventing the disease from occurring in a mammal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease), such as by disruption of pre-formed amyloid or synuclein fibrils. One such preventive treatment may be use of the disclosed compounds for the treatment of Mild Cognitive impairment (MCI).

“Fibrillogenesis” refers to the formation, deposition, accumulation and/or persistence of amyloid fibrils, filaments, inclusions, deposits, as well as synuclein (usually involving α-synuclein) and/or NAC fibrils, filaments, inclusions, deposits or the like.

“Inhibition of fibrillogenesis” refers to the inhibition of formation, deposition, accumulation and/or persistence of such amyloid fibrils or symiclein fibril-like deposits.

“Disruption of fibrils or fibrillogenesis” refers to the disruption of pre-formed amyloid or synuclein fibrils, that usually exist in a pre-dominant β-pleated sheet secondary structure. Such disruption by compounds of the invention may involve marked reduction or disassembly of amyloid or synuclein fibrils as assessed by various methods such as circular dichroism spectroscopy, Thioflavin T fluorometry, Congo red binding, SDS-PAGE/Western blotting, as demonstrated by the Examples presented in this application.

“A pharmaceutical agent” or “pharmacological agent” or “pharmaceutical composition” refers to a compound or combination of compounds used for treatment, preferably in a pure or near pure form. In the specification, pharmaceutical or pharmacological agents include the compounds of this invention. The compounds are desirably purified to 80% homogeneity, and preferably to 90% homogeneity. Compounds and compositions purified to 99.9% homogeneity are believed to be advantageous. As a test or confirmation, a suitable homogeneous compound on HPLC would yield, what those skilled in the art would identify as a single sharp-peak band.

### Compounds of the Invention

The compound of this invention is 3,4-dihydroxybenzoic acid 3,4-dihydroxyanilide in a SMEDD formulation and the pharmaceutically acceptable salts of the compound.

### Synthesis of the Compound of the Invention and Formulations

The compound of this invention may be prepared by methods generally known to the person of ordinary skill in the art, having regard to that knowledge and the disclosure of this application including Examples 1-10.

The starting materials and reagents used in preparing these compounds are either available from commercial suppliers such as the Aldrich Chemical Company (Milwaukee, Wis.), Bachem (Torrance, Calif.), Sigma (St. Louis, Mo.), or Lancaster Synthesis Inc. (Windham, N.H.) or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

Other starting materials or early intermediates may be prepared by elaboration of the materials listed above, for example, by methods well known to a person of ordinary skill in the art.

The starting materials, intermediates, and compounds of this invention may be isolated and purified using conventional techniques, including precipitation, filtration, distillation, crystallization, chromatography, and the like. The compounds may be characterized using conventional methods, including physical constants and spectroscopic methods.

### Pharmacology and Utility

The compounds of this invention, either as the dihydroxyaryl compounds per se, or as the methylenedioxy analogs or pharmaceutically acceptable esters (once de-protected either in the body or in vitro), act to inhibit or prevent amyloid fibril formation, inhibit or prevent amyloid fibril growth, and/or cause disassembly, disruption, and/or disaggregation of pre-formed amyloid fibrils and amyloid protein deposits. Their activity can be measured in vitro by methods such as those discussed in the Examples, while their activity in vivo against systemic AA amyloid diseases can be measured in animal models, that mimic many of the neuropathological hallmarks of systemic AA amyloid disease.

“Amyloid diseases” or “amyloidoses” suitable for treatment with the compounds of this invention are diseases associated with the formation, deposition, accumulation, or persistence of amyloid fibrils, especially the fibrils of an AA amyloid protein. Suitable such diseases include, the amyloidosis of chronic inflammation, the amyloidosis of malignancy and Familial Mediterranean Fever.

### Pharmaceutical Compositions and Administration

In general, compounds of the invention will be administered in therapeutically effective amounts by any of the usual modes known in the art, either singly or in combination with at least one other compound of this invention and/or at least one other conventional therapeutic agent for the disease being treated. A therapeutically effective amount may vary widely depending on the disease, its severity, the age and relative health of the animal being treated, the potency of the compound(s), and other factors. As anti-fibril agents, therapeutically effective amounts of compounds of this invention may range from 0.1-1000 mg/Kg body weight/day, such as from 1-100 mg/Kg/day; for example, 10-100 mg/Kg/day. A person of ordinary skill in the art will be conventionally able, and without undue experimentation, having regard to that skill and to this disclosure, to determine a therapeutically effective amount of a compound for the treatment of an AA amyloid disease.

In general, the compounds of this invention will be administered as pharmaceutical compositions by one of the following routes: oral, topical, systemic (e.g. transdermal, intranasal, or by suppository), or parenteral (e.g. intramuscular, subcutaneous, or intravenous injection). Compositions may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references as Remington: The Science and Practice of Pharmacy, A. Gennaro, ed., 20th edition, Lippincott, Williams & Wilkins, Philadelphia, Pa. Suitable liquid carriers, especially for injectable solutions, include water, aqueous saline solution, aqueous dextrose solution, and glycols.

In particular, the compound can be administered, orally, for example, as tablets, troches, lozenges, aqueous or oily suspension, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Oily suspensions may be formulated by suspending the compound in a vegetable oil, for example arachis oil, olive oil, sesame oil, or coconut oil or in a mineral oil such as liquid paraffin. The oily suspensions may contain a thickening agent, for example beeswax, hard paraffin or cetyl alcohol. Sweetening agents, such as those set forth below, and flavoring agents may be added to provide a palatable oral preparation. These compositions may be preserved by the addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already described above. Additional excipients, for example sweetening, flavoring and agents, may also be present.

The compounds of the invention may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally occurring phosphatides, for example soy bean, lecithin, and occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsion may also contain sweetening and flavoring agents. Syrups and elixirs may be formulated with sweetening agents, for example, glycerol, sorbitol or sucrose. Such formulations may also contain a demulcent, a preservative and flavoring and coloring agents.

Other oral delivery systems such as self-microemulsifying drug delivery systems (SMEDDS) in liquid and pellet forms that result in improved solubility, dissolution, and in vivo oral absorption of the poorly water-soluble compounds can be formulated such as those developed for curcumin. (European Journal of Pharmaceutics and Biopharmaceutics (2010), 76: 475-485).

It is especially advantageous to formulate the compounds in dosage unit form for ease of administration and uniformity of dosage. Dosage unit form as used herein refers to physically discrete units suited as unitary dosages for the subjects to be treated; each containing a therapeutically effective quantity of the compound and at least one pharmaceutical excipient. A drug product will comprise a dosage unit form within a container that is labeled or accompanied by a label indicating the intended method of treatment, such as the treatment of an amyloid disease, for example an amyloidosis such as Alzheimer's disease or a disease associated with α-synuclein/NAC fibril formation such as Parkinson's disease.

### EXAMPLES

### Example 1

### 3,4-dihydroxybenzoic acid 3,4-dihydroxyanilide (Compound 51; DC-0051)

### Method 1—via Methylenedioxy-Protected Compounds

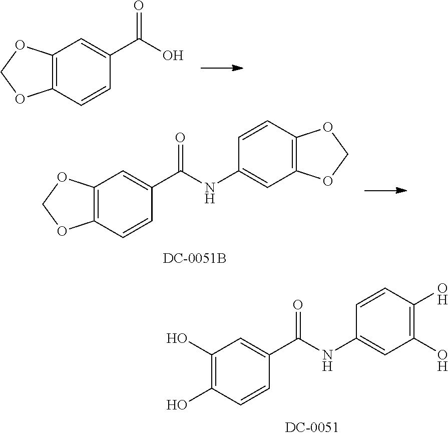 [SMILES: O=C(O)c1ccc2c(c1)OCO2 ; O=C(Nc1ccc2c(c1)OCO2)c1ccc2c(c1)OCO2 ; O=C(Nc1ccc(O)c(O)c1)c1ccc(O)c(O)c1]

### 3,4-methylenedioxybenzoic acid 3,4-methylenedioxyanilide (Compound 51)

To a solution of piperonylic acid (500 mg, 3 mmol) in dry CH₂Cl₂ (25 mL) under nitrogen, was added oxalyl chloride (573 mg, 4.5 mmol) with three drops of dry DMF, and the mixture was stirred for 1 hour. Solvents were removed in vacuo giving the acid chloride as a white solid. To a solution of the acid chloride in dry CH₂Cl₂ (50 mL) under nitrogen, cooled to 0° C., was added dropwise, a solution made up of 3,4-(methylenedioxy)aniline (498 mg, 30.1 mmol) and pyridine (0.5 mL) in CH₂Cl₂ (5 mL). The reaction mixture was stirred for 30 minutes at room temperature, then diluted by the addition of CH₂Cl₂ (100 mL), washed with aqueous HCl (50 mL, 10%) and sodium bicarbonate solution (50 mL) then dried. Solvents were removed in vacuo to give the crude product as a brown crystalline material. Recrystallization from aqueous ethanol gave DC-0051B as small silvery crystals (0.516 g, 60%).

### ¹H-NMR(CDCl₃) 7.60 (1H, br s), 7.35 (3H, m), 6.88 (2H, m), 6.78 (1H, d, j 9Hz), 6.06 (2H, s) and 5.98 (211, s).

### 3,4-dihydroxybenzoic acid 3,4-dihydroxyanilide (compound 51; DC-0051)

To a solution of DC-0051B (100 mg) in dry CH₂Cl₂ (25 mL) under nitrogen was added BBr₃ (0.2 mL) and the mixture was stirred for 6 hours. After stirring, aqueous 3M HCl (25 mL) was carefully added to the reaction mixture. The product was extracted into EtOAc (200 mL), dried and evaporated in vacuo to give the crude product. Purification by column chromatography (Silica:Hexane/EtOAc 30:70) gave DC-0051 as an off-white solid (71 mg, 77%). ¹H-NMR(CD₃OD) 7.60 (1H, br s), 7.38 (1H, d, J 2 Hz), 7.33 (1H, dd, J 2, 8 Hz), 7.21 (1H, d, J 2 Hz), 6.89 (1H, dd, J 2, 8Hz), 6.86 (1H, d, J 8 Hz) and 6.76 (11-1, d, J 8 Hz). M/z 262 ((M+1)⁺, 100%) HPLC (method 2) 15.1 minutes.

### Method 2—via Benzyloxy- and Methoxymethoxy-Protected Compounds:

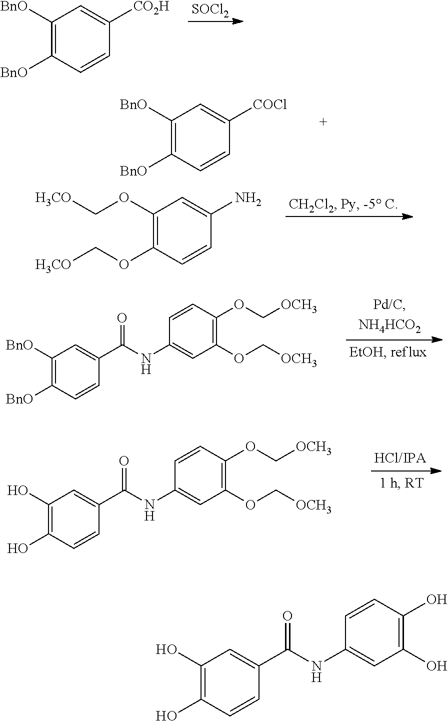

### 3,4-dibenzyloxybenzoyl chloride

3,4-dibenzyloxybenzoic acid (3.1 g. 9.3 mmol) was combined with pyridine (5 drops, catalytic) and thionyl chloride (15 mL, 205 mmol). The solution was heated at reflux for 4 h, cooled, and excess thionyl chloride removed under reduced pressure. The crude product was dissolved in benzene (50 mL), and stripped of solvent under vacuum. The benzoyl chloride (theoretical yield 3.4 g) was then dissolved in dichloromethane and used directly in the next step.

### 3,4-dibenzyloxybenzoic acid 3,4-di(methoxymethoxy)anilide

3,4-di(methoxymethoxy)aniline (0.484 g, 2.2 mmol) was dissolved in dichloromethane (5 mL) and pyridine (3 mL) and cooled to −5° C., while stirring under nitrogen. A solution of 3,4-dibenzyloxybenzoyl chloride in dichloromethane (0.8 g, 2.2 mmol of acid chloride) was added dropwise over 30 minutes. The reaction was allowed to stir at 0° C. for 30 minutes then warmed to room temperature over 30 minutes. The reaction was diluted with dichloromethane (100 mL), washed with aqueous citric acid (3×300 mL of a 2% w/v solution), aqueous sodium hydroxide (2×35 mL of a 2% w/v solution) and dried (Na₂SO₄). Removal of the solvent under reduced pressure afforded a solid, 0.97 g. The crude product was triturated with warm methanol (10 mL) and filtered to afford the desired product, 0.5 g.

### 3,4-dihydroxybenzoic acid 3,4-di(methoxymethoxy)anilide

3,4-dibenzyloxybenzoic acid 3,4-di(methoxymethoxy)benzanilide (0.2 g, 0.4 mmol) was combined with ethanol (10 mL), and palladium on charcoal (40 mg of 10% Pd/C). The reaction was heated to reflux with stirring under nitrogen, and ammonium formate (0.8 g, 12.7 mmol) was added portion wise over 15 min and then held at reflux for two hours. The cooled reaction solution was filtered to remove the catalyst and concentrated under reduced pressure to afford the crude product, 0.13 g.

### 3,4-dihydroxybenzoic acid 3,4-dihydroxyanilide (Compound 51; DC-0051)

3,4-dihydroxybenzoic acid 3,4-di(methoxymethoxy)benzanilide (0.17 g, 0.49 mmol) was combined with a 25% solution of hydrogen chloride in isopropyl alcohol (15 mL) and water (1 mL). The reaction was stirred at room temperature for 1 h and the solvent removed under reduced pressure. Trituration with diethyl ether (5 mL) afforded DC-0051 as a solid which was dried under vacuum at 30° C., yield 60 mg.

### Example 2

### Preparation of Amyloid Enhancing Factor (AEF)

On Day 1 the spleens of mice previously induced with AEF were selected for the prescence of amyloid and weighed (Gervais, F et al., J. Leuk. Bio. (1988) 43:311-316 and Hol, P. R. et al., Br. J. Exp. Path (1985)66:689-97). The spleens were then transfered to a Kontes grinder and homogenized in 31 mL of 0.9% NaCl (Saline) until slurry. The slurry was entrifuged at 10,000 RPM for 30 minutes and the supernatant was discarded. The pellet was re-homogenize in 31 mL Saline which was repeated 5 times. The pellet was stored at 4° C. overnight. On day 2 the pellet was resuspended in 23 mL ddH₂O to remove salt and centrifuged at 15,000 RPM for 2 hours. The pellet was resuspended in 15 mL ddH₂O and again centrifuged at 15,000 RPM for 2 hours. The supernatant was saved and labelled Sup II. This step was repeated two more times labeling subsequent supernatents as Sup III, and Sup IV respectively. On Day 3 500 uL of each saved supernatant for use in a Bradford Asssay for protein determination. Sup II, Sup III, and Sup IV were pooled and 1 mL was aliquoted into a tube and lyophilized and the material weighed.

### Example 3

### Induction of Mice with AEF to Create Experimental AA Amyloid Mouse Model

The AEF preparation was delivered on day (minus) −14 of dosing by lateral tail vein injection of 80 μg/100 μL in sterile water. Concominent with AEF, a 0.5 mL subcutaneous injection of 3% silver nitrate solution was delivered to each mouse between the scapulae. Mice were observed each day for adverse reaction to this procedure.

### Example 4

### SMEDDs Preparation and Formulation of Compound 51

This protocol is derived from the publication by Setthacheewakul, et al. where the absorption and PK of curcumin was evaluated using different SMEDDS “self-microemulsifying drug delivery system” formulations (Setthacheewakul, S., et al., Eur. J. Pharm. Biopharm., 2010, 76: 475-485).

This protocol describes how to prepare an 80 mg/mL (in ˜30% oil/˜70% surfactant) stock formulation. The stock formulation is diluted 4-fold with DI water to make a 20 mg/mL final concentration for dosing.

This total protocol requires 48 hours before the stock solution can be diluted and used for dosing. Remove Compound 51 from 4° C. to room temperature and allow the compound to reach room temperature over 20-30 minutes. Weigh 800 mg Compound 51 and place in 14 mL polypropylene tube. Dissolve Compound 51 in the oil mixture first: Tare the tube containing Compound 51, and weigh into the tube 1.35 grams of Capryol 90 (GatteFosse) and 1.35 grams Labrafac PG (GatteFosse) (oil phase). Next, using a pipet, add the two oils drop wise, weighing the 1.35 grams of each oil into the tube containing Compound 51.

Place on Vortexer for 40 minutes or until a homogenous mixture is obtained. Prepare the surfactant mixture in separate 14 mL polypropylene tube: tare and weigh into that tube 3.15 grams of Solutol and 3.15 grams Labrasol (GatteFosse)(surfactant phase). Add the Labrasol drop wise on top of the Solutol in the round bottom tube. Place the Solutol/Labrasol mixture at 37° C. for approximately 20 minutes to make a homogenous mixture of the surfactants. Allow the oil and surfactant mixtures to equilibrate separately overnight at room temperature.

The next morning, warm both solutions at 37° C. for approximately 30 minutes. The surfactant phase (Solutol/Labrasol) should be clear. The Compound 51/oil will be lavender colored and will likely appear in two layers. Spin at 2000 rpm in to pull as much material into the bottom of the mixtures as possible. Gently vortex the Compound 51/oil mixture and transfer the Solutol/Labrasol mix into the Compound 51/oil mix by adding the surfactant mixture to the Compound 51/oil mixture. Once the transfer is complete, vigorously vortex the mixture. Vortex for 2 hours, and allow the mixture to sit overnight at room temperature.

The solution is now ready to make into dosing formulation and should appear completely homogenous. Overnight, the solution may have settled (dark purple thin bottom layer and light purple/cloudy large top layer) and will likely require additional vortexing (tape to vortex for 10-30 minutes) prior to preparation of the dosing solution. To make the final dosing solution, a 1:4 dilution is made (e. g. add 1 mL of 80 mg/mL Compound 51 oil/surfactant solution to 3 mL of DI water) to make an oil-in-water emulsification with a final Compound 51 concentration of 20 mg/mL. In order to obtain a mostly clear (but slightly cloudy) dosing solution with the micelles, 5-20 μL of Labrafac per mL of dosing solution is added as the final step.

### Example 5

### Animal Dosing, Sacrifice and Harvest

CBA/J female mice approximately 25 weeks old were randomly assigned to four groups for the following treatment options:

Group #1—2 month dosing=Compound 51+SMEDDs Oral 100 mg/kg N=10

Group #2—2 month dosing=Compound 51+SMEDDs Oral 50 mg/kg N=10

Group #3—2 month dosing=Compound 51+SMEDDs Oral 25 mg/kg N=10

Group #4—2 month dosing=SMEDDs Vehicle Control N=10 Oral dosing was begun at 100 mg/kg, 50 mg/kg and 25 mg/kg, two weeks after induction of amyloidosis and was continued for a further eight weeks. Oral doses were formulated in an oil/surfactant self-microemulsifying delivery system one day prior to dosing by gavage. Oral gavages were achieved using Popper and sons blunt end mouse gavage needles I.P. dosing was achieved with a 27G hypodermic needle.

On Day 60 (2 months after beginning oral dosing) all mice were euthanized by CO₂ overdose. The spleen, liver and kidney were fixed in 4% Para formaldehyde for 24 hours and sent to an independent lab for paraffin processing.

### Example 6

### Compound 51 in SMEDDS Formulation Causes a Marked Reduction in Pre-Existing AA Amyloid Deposits in Liver as Assessed by Congo Red Fluorescence

Congo Red Staining Protocol, treat the Paraffin sections to deparaffinize and hydrate to dH2O. Soak briefly ˜5 seconds in distilled water. Rinse with distilled water ˜3-4 changes. Add 200 mL Alkaline salt solution (80% EtOH saturated with NaCl) into staining dish for 25 min. Add 2.0 mL of 1% Sodium Hydroxide to salt solution. Filter 200 mL Congo red Solution (see formulation below) prior to use. Add 2.0 mL of 1% Sodium Hydroxide to Congo red solution for 25 min. Dip quickly into 80% ETOH—dip quickly 100% EtOh, twice—then dip into Xylene 3X. Permount and coverslip.

Stock Congo Red Solution. Dissolve 4 g Congo Red dye in 400 mL distilled water. Add 1600 mL 100% EtOh and stir, then add 40 g NaCl3 and stir. Store stock solution in fridge—foil covered (filter before use). This Congo red staining protocol was used to obtain all the data presented herein.

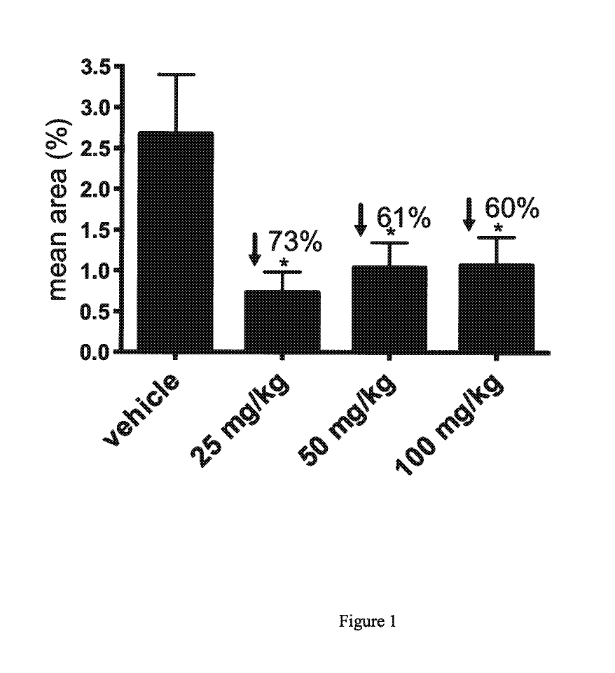

FIG. 1 illustrates that a 25 mg/kg/day dose of compound 51 in the SMEDDs formulation significantly reduced by 73% the amount of amyloid in the liver of mice induced with AEF as assessed by Congo Red Fluorescence. A corresponding dose of 50 mg/kg/day of compound 51 in the SMEDDs formulation showed a 61% reduction, while a dose of 100 mg/kg/day of compound 51 in the SMEDDs formulation showed a 60% reduction. In the graph outliers (0-1 per group) were identified by Grubbs' outlier test and excluded. P*<0.05 by one-way ANOVA and Dunnet's post-hoc test. In this experiment, N=9-10 per group.

### Example 7

### Compound 51 in SMEDDS Formulation Causes a Marked Reduction in Pre-Existing AA Amyloid Deposits in Kidney as Assessed by Congo Red Fluorescence

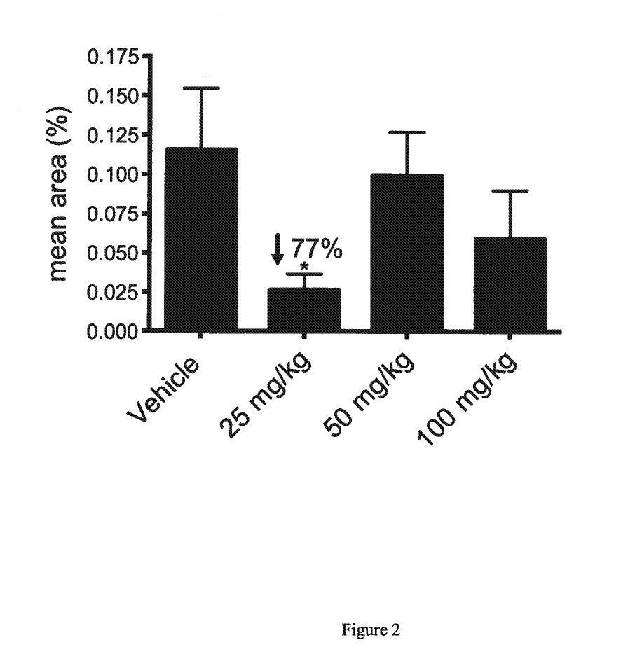

FIG. 2 illustrates that a 25 mg/kg/day dose of compound 51 in the SMEDDs formulation significantly reduced by 77% the amount of amyloid in the kidney of mice induced with AEF as assessed by Congo Red Fluorescence. In the graph outliers (0-1 per group) were identified by Grubbs' outlier test and excluded. P*<0.05 by two-tailed student's t-test. In this experiment, N=9-10 per group.

### Example 8

### Compound 51 in SMEDDS Formulation Causes a Marked Reduction in Pre-Existing AA Amyloid Deposits in Spleen as Assessed by Congo Red Fluorescence

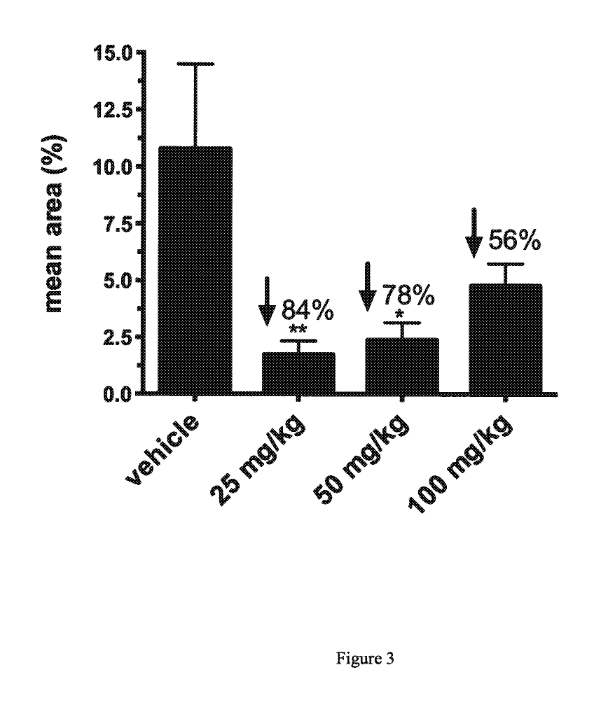

FIG. 3 illustrates that a 25 mg/kg/day dose of compound 51 in the SMEDDs formulation significantly reduced by 84% the amount of amyloid in the spleen of mice induced with AEF as assessed by Congo Red Fluorescence. A corresponding dose of 50 mg/kg/day of compound 51 in the SMEDDs formulation showed a 78% reduction, while a dose of 100 mg/kg/day of compound 51 in the SMEDDs formulation showed a 56% reduction. In the graph outliers (0-1 per group) were identified by Grubbs' outlier test and excluded. P**<0.01, P*<0.05 by one-way ANOVA and Dunnet's post-hoc test. In this experiment, N=9-10 per group.

### Example 9

### Compound 51 in SMEDDS Formulation Causes a Marked Reduction in Pre-Existing AA Amyloid Deposits in Liver as Assessed by anti-AA Immunostaining

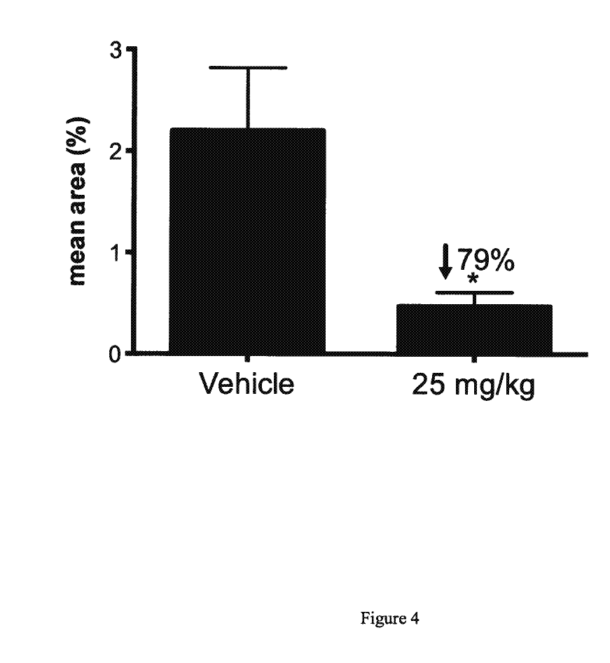

Using standard immunostaining protocols, the paraffin sections of liver were immunostained with anti-AA antibodies to quantify any reduction in AA amyloid load in the liver of AEF induced, mice treated with Compound 51. FIG. 4 illustrates that a 25 mg/kg/day dose of compound 51 in the SMEDDs formulation significantly reduced by 79% the amount of amyloid in the liver of mice induced with AEF as assessed by anti-AA Immunostaining.

### Example 10

### Compound 51 in SMEDDS Formulation Causes a Marked Reduction in Pre-Existing AA Amyloid Deposits in Kidney as Assessed by Anti-AA Immunostaining

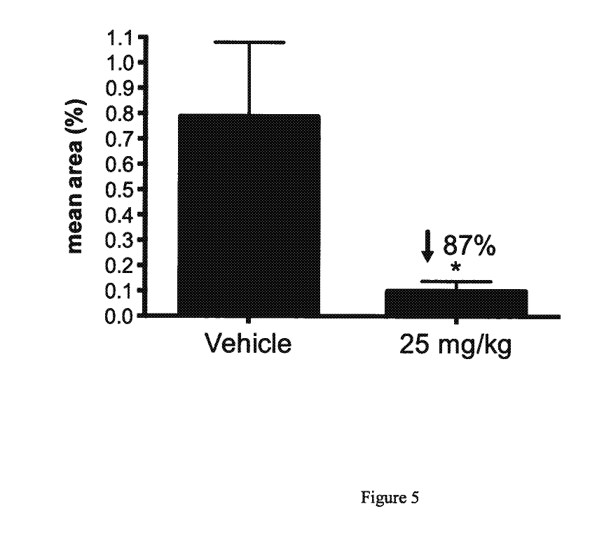

Using standard immunostaining protocols, the paraffin sections of kidney were immunostained with anti-AA antibodies to quantify any reduction in AA amyloid load in the kidney of AEF induced, in mice treated with Compound 51. The antibody used was the monoclonal Anti-Human Amyloid A MCA1862Ht clone mc1 from Serotec. FIG. 5 illustrates that a 25 mg/kg/day dose of compound 51 in the SMEDDs formulation significantly reduced by 87% the amount of amyloid in the kidney of mice induced with AEF as assessed by anti-AA Immunostaining. In the graph outliers (0-1 per group) were identified by Grubbs' outlier test and excluded. P*<0.05 by two-tailed student's t-test. In this experiment, N=9-10 per group.

We claim:

1. A pharmaceutical composition comprising 3,4-dihydroxybenzoic acid 3,4-dihydroxyathlide, an oil and a surfactant.

2. The composition of claim 1 wherein the oil is mixture of Capryol 90 and Labrafac PG.

3. The composition of claim 1 wherein the surfactant is a mixture of Labrasol and Solutol HS 15.

4. The composition of claim 1 where the proportion of oil to surfactant is 30%:70%.

5. A method of treating the formation, deposition, accumulation, or persistence of AA amyloid fibrils, comprising treating the fibrils with an effective amount of the composition of claim 1.

6. A method of inhibiting and/or relieving an AA amyloid disease in a mammal suffering therefrom, comprising administration to the mammal of a therapeutically effective amount of the composition of claim 1.

7. The method of claim 1, wherein the mammal is a human.

8. The method of claim 1, wherein the amount of the composition administered is between 1 mg/Kg/day and 100 mg/Kg/day.

9. The method of claim 1, wherein the amount of composition administered is between 10 mg/Kg/day and 50 mg/Kg/day.
